(19) United States
(12) Patent Application Publication
(10) Pub. No.: US 2006/0184081 A1
(43) Pub. Date: Aug. 17, 2006

(54) Enhanced arm brace

(76) Inventor: Robert Farrer Gilmour, Auckland (NZ)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 10/515,727

(22) PCT Filed: May 28, 2003
(86) PCT No.: PCT/NZ03/00107

§ 371 Date: Mar. 30, 2006

(30) Foreign Application Priority Data
May 28, 2002 (NZ) 519192

(51) Int. Cl. A61F 5/00

(52) U.S. Cl. 602/20; 602/5

(57) ABSTRACT

A brace (1) with one or more shells (2, 3) to engage a user's arm, a hand post (10), and adjustable engagement elements (25, 29) between the hand post and the shell which provides a plurality of selectable positions between the hand post and the shell to enable the configuration of the brace to be altered.

### TECHNICAL FIELD

This invention relates to a brace, and in particular an orthopaedic arm brace.

### BACKGROUND ART

Arm braces which control motion of the elbow of the user and which hold the upper and lower arm in hard shells are known. There are also devices available which are able to act as a sling, devices that prevent shoulder rotation and abduction, devices that control wrist position in multiple planes, and devices which control the proximal radioulnar joint whereby pronation and supination rotation of the forearm occurs at the prox radioulnar joint at the elbow where the head of the radius rotates in a groove on the ulnar bone. Humeral fracture braces are also known. By using Plaster of Paris many, if not all, of these functions can be performed, but Plaster of Paris has disadvantages in that it is not readily fitted and removed.

Accordingly the present position is disadvantageous.

### OBJECT OF THE INVENTION

It is therefore an object of the present invention to provide a brace which will obviate or minimise the foregoing disadvantages in a simple yet effective manner or which will at least provide that public with a useful choice.

### STATEMENT OF THE INVENTION

Accordingly in one aspect the invention consists in a brace comprising or including a shell to engage a user's arm, a hand post, and adjustable engagement means between the hand post and the shell, the engagement means providing a plurality of selectable positions between the hand post and the shell to enable the configuration of the brace to be altered.

Preferably the shell has a longitudinal axis and the hand post is able to be positioned in a plurality of positions in a plane transverse to the longitudinal axis of the shell.

Preferably three positions are provided on the shell for attachment of the hand post.

Preferably in at least one of said plurality of said selectable positions, between the hand post and the shell, the hand post is able to be positioned in a selected one of a plurality of available positions at selected angles to the longitudinal axis of the shell.

Preferably at least one of said angles is such that the hand post lies parallel to the longitudinal axis of the shell.

Preferably three available angular angles are provided.

Preferably the shell comprises two shell elements, one to engage the forearm and one to engage the upper arm.

To those skilled in the art to which the invention relates, many changes in construction and widely differing embodiments and applications of the invention will suggest themselves without departing from the scope of the invention as defined in the appended claims. The disclosures and the description herein are purely illustrative and are not intended to be in any sense limiting.

### BRIEF DESCRIPTION OF THE DRAWINGS

One preferred form of the invention will now be described with reference to the accompanying drawings in which,

FIG. 1 is an underneath view of a brace according to one preferred form of the invention,

FIG. 2 is a plan view of the brace of FIG. 1 but with the hand post in an alternative position,

FIG. 3 is a further view as in FIG. 2 with the hand post in a still further position,

FIG. 4 is a part view of the underneath of the brace of FIG. 1 with the hand post in a first rotated position,

FIG. 5 shows an alternative rotated position, and

FIG. 6 shows a still further rotated position.

### BEST MODE FOR CARRYING OUR THE INVENTION

Referring to the drawings, a brace 1 is provided which includes a shell preferably made up of two shell parts 2 and 3. The shell 2 is able to engage the forearm of a user, and the shell 3 the upper part of the arm of a user. A user's arm is held in the shells in any available and useful manner such as by the use of wrapping 30 and straps 21 shown about shell 3.

The parts 2 and 3 may be connected by one or more range of motion devices 4. In the construction of the figures, two such range of motion devices are provided between the shell elements 2 and 3. Range of motion devices are known and any selected range of motion device may be used.

Thus each range of motion machine may be connected to an arm 5 which engages the shell element 2 and an arm 6 which engages the shell element 3.

The shell elements are provided with attachment devices to enable them to be held against the user's arms, for example, in the known manner.

The brace includes a hand post 10, which hand post 10 may be provided by a substantially rigid element 11 which is formed substantially into an “L” shape and extends outwardly from the shell element 2.

The free end of the element 11 may carry a connection device such as a ring 12 onto which, for example, a strap 13 which may form an arm sling, may be connected.

The strap 13 is adjustable in length, for example, by providing a returned part 15 at the other end which passes through a locating guide 16.

The arm 11 may be surrounded by a cushioning grip element 14 formed, for example, of a foamed rubber or plastic material so as to provide comfort for the user's hand.

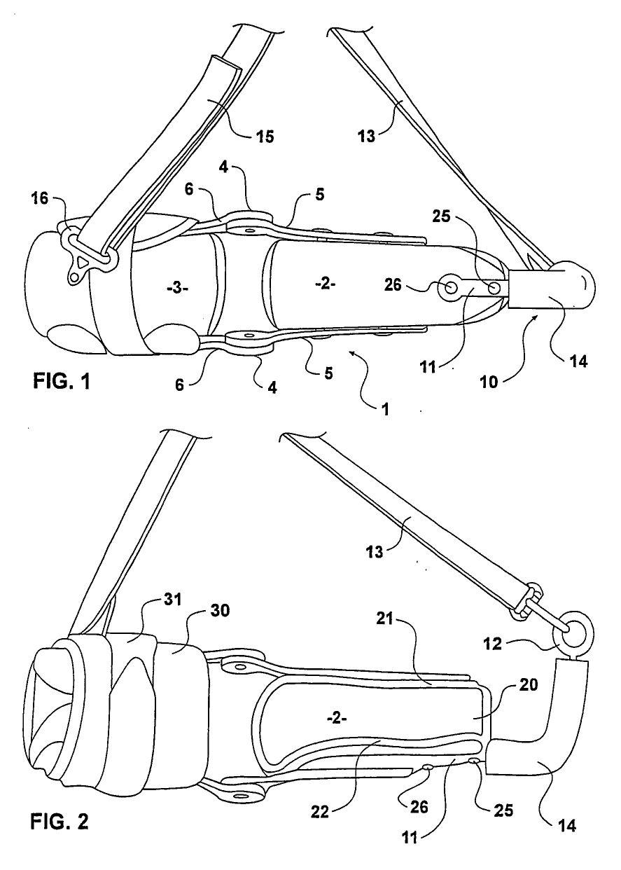

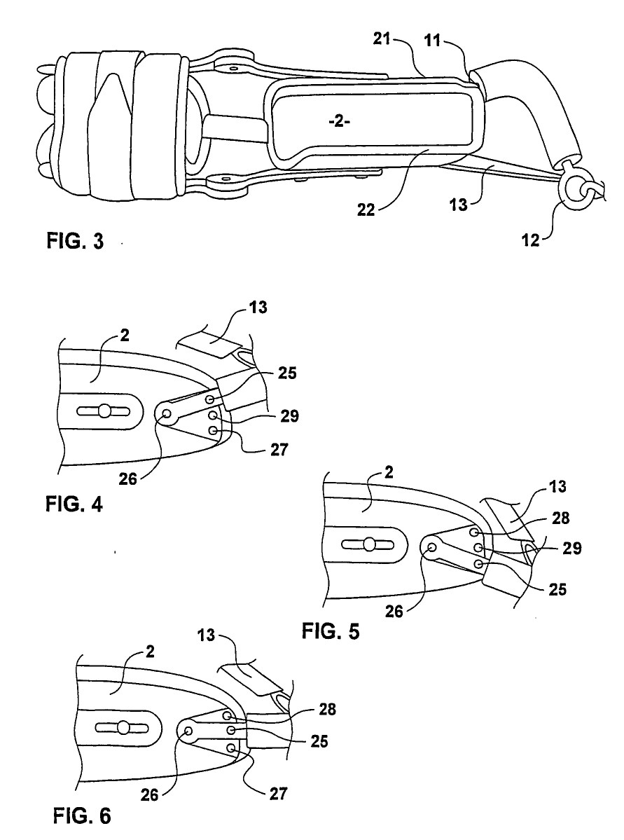

The hand post 10 is engaged with the shell element 2 in any one of a selected plurality of available positions. Thus the shell element 2 may have a base part 20, an inner side wall 21 and an outer side wall 22. In the preferred embodiment of the invention the hand post can be connected to any one of the base part 20 as shown in FIG. 1, the outer wall 22 as shown in FIG. 2, and the inner wall 21 as shown in FIG. 3.

Connection can be in any desired manner, for example, by providing a pair of apertures at each location position into which fixing screws 25 and 26 are able to be positioned. The connection can be varied simply by removing the fixing screws 25 and 26, repositioning the hand post 10 to another available position and reconnecting the fixing screws 25 and 26.

Furthermore in at least one of the positions, more desirably both side positions, and if desired all three of the positions, a further flexibility in the design is provided by enabling the angle between the member 11 and the longitudinal axis of the shell element 2 to be varied.

In the preferred form this is achieved by providing further fixing apertures 27 and 28 each side of a central aperture into which the screw 25 is positioned in FIGS. 2 and 3. Thus, for example, in FIG. 4 the screw 25 is positioned into aperture 28 leaving apertures 27 and 29 empty. In FIG. 5 the apertures 28 and 29 are empty and the screw 25 is positioned in aperture 27. In FIG. 6 the apertures 28 and 27 are empty and the screw 25 is positioned in aperture 29.

Thus the hand post can be positioned in a neutral position or in positions adjusted for flexion and extension. FIGS. 4, 5 and 6 are essentially side elevations of the construction as shown in FIG. 2, and FIGS. 2 and 3 show pronated and supinated positions of the hand post.

Accordingly, and in particular, the pronated and supinated positions can be adjusted for flexion and extension.

It will be clear that tension can be applied to the hand post control strap to create a resting position in ulna or radial deviation.

Thus it can be seen that a brace is provided which, because of the substantial range of positions available for the hand post, is able to achieve the variations unable to be achieved by Plaster of Paris on the arm, yet in a way that the brace can be readily fitted, removed to allow adjustment of the configuration, and then refitted.

Thus at least in the preferred form of the invention a brace is provided wherein the hand post attachment to he forearm shell can be varied in position. It can remain in a neutral position or, in particular, it can be fitted to either side of the shell, and in particular the shell element attached to the forearm of the user to provide a position of supination or pronation. Also the brace has provision for variation in the attachment point to allow for wrist flexion and extension to be incorporated into the supinated or pronated position. Further the hand post has flexibility and the hand post strap allows the wrist to be held in variable degrees of ulna and radial deviation.

What we claims is:

1. A brace comprising or including a shell to engage a user's arm, a hand post, and adjustable engagement means between the hand post and the shell, the engagement means providing a plurality of selectable positions between the hand post and the shell to enable the configuration of the brace to be altered.

2. A brace as claimed in claim 1 wherein the shell has a longitudinal axis and the hand post is able to be positioned in a plurality of positions in a plane transverse to the longitudinal axis of the shell.

3. A brace as claimed in claim 2 wherein three positions are provided on the shell for attachment of the hand post.

4. A brace as claimed in claim I wherein in at least one of said plurality of said selectable positions, between the hand post and the shell, the hand post is able to be positioned in a selected one of a plurality of available positions at selected angles to the longitudinal axis of the shell.

5. A brace as claimed in claim 4 wherein at least one of said angles is such that the hand post lies parallel to the longitudinal axis of the shell.

6. A brace as claimed in claim 4 wherein three available angular angles are provided.

7. A brace as claimed in claim 1 wherein the shell comprises two shell elements, one to engage the forearm and one to engage the upper arm.
